(19) United States
(12) Patent Application Publication
(10) Pub. No.: US 2014/0121559 A1
(43) Pub. Date: May 1, 2014

(54) DETECTING COGNITIVE IMPAIRMENT INDICATORS

(71) Applicant: INTERNATIONAL BUSINESS MACHINES CORP, Armonk, NY (US)

(72) Inventors: Mark B. Stevens, Austin, TX (US); John D. Wilson, Houston, TX (US)

(73) Assignee: INTERNATIONAL BUSINESS MACHINES CORPORATION, Armonk, NY (US)

(21) Appl. No.: 13/666,677

(22) Filed: Nov. 1, 2012

(51) Int. Cl. A61B 5/16; A61B 5/12; A61B 5/00

(52) U.S. Cl. 600/558; 600/300; 600/559

(57) ABSTRACT

A method, system or computer usable program product for monitoring user interaction patterns to provide early warning of cognitive impairment including monitoring with an application running on a processor a first set of user interactions of a user with a data processing system to form a historical baseline for the user that is stored in memory, monitoring with the application running on the processor a second set of user interactions of the user with the data processing system to form a set of current results for the user, comparing with the processor the set of current results with the historical baseline, and responsive to identifying a change indicative of a cognitive degradation for the user, sending a notification indicating the change.

### BACKGROUND

1. Technical Field

The present invention relates generally to detecting user interaction patterns indicating cognitive impairment, and in particular, to a computer implemented method for monitoring and tracking user interaction patterns to provide early warning of cognitive impairment.

2. Description of Related Art

Cognitive impairment can develop slowly over many years with the case of Alzheimer's disease or dementia or can have acute onset due to head trauma or other disease conditions. In either case, the patient may not recognize the symptoms, may discount the degree of impairment, or the symptoms of early onset may be subtle.

Research indicates that dementia and head trauma effect reaction time, memory, and the ability to perform complex tasks. In the case of head trauma, the change in performance abilities is sudden and significant. For chronic debilitation as occurs in various forms of dementia the performance degradation occurs slowly over time. Today, hockey player routinely have a concussion baseline test performed before the season starts that measures reaction and memory performance. If a head injury is suspected, the test is re-done and measured against the baseline.

This type of testing is not routine and in many cases it would not be prescribed because changes to performance may not be noticed until performance is grossly affected. It is said that Alzheimer's disease is typically diagnosed six years after onset. This is unfortunate because there is research that indicates that early intervention with Alzheimer's disease can prolong the period of higher functional performance.

### SUMMARY

The illustrative embodiments provide a method, system, and computer usable program product for monitoring user interaction patterns to provide early warning of cognitive impairment including monitoring with an application running on a processor a first set of user interactions of a user with a data processing system to form a historical baseline for the user that is stored in memory, monitoring with the application running on the processor a second set of user interactions of the user with the data processing system to form a set of current results for the user, comparing with the processor the set of current results with the historical baseline, and responsive to identifying a change indicative of a cognitive degradation for the user, sending a notification indicating the change.

### BRIEF DESCRIPTION OF THE SEVERAL VIEWS OF THE DRAWINGS

The novel features believed characteristic of the invention are set forth in the appended claims. The invention itself, further objectives and advantages thereof, as well as a preferred mode of use, will best be understood by reference to the following detailed description of illustrative embodiments when read in conjunction with the accompanying drawings, wherein:

FIG. 1 is a block diagram of a data processing system in which various embodiments may be implemented;

FIG. 2 is a block diagram of a network of data processing systems in which various embodiments may be implemented;

FIG. 3 is a high level flow diagram of user interaction monitoring in which various embodiments may be implemented;

FIG. 4 is a block diagram of a single system monitoring implantation in accordance with a first embodiment;

FIG. 5 is a flow diagram of the operation of the single system monitoring implementation in accordance with the first embodiment;

FIG. 6 is a block diagram of a multi-system monitoring implantation in accordance with a second embodiment;

FIG. 7 is a flow diagram of the operation of the multi-system monitoring implementation in accordance with the second embodiment; and

FIG. 8 is a block diagram of a monitoring parameters database in which various embodiments may be implemented.

### DETAILED DESCRIPTION

Processes and devices may be implemented and utilized to monitor and track user interaction patterns to provide early warning of cognitive impairment. These processes and apparatuses may be implemented and utilized as will be explained with reference to the various embodiments below.

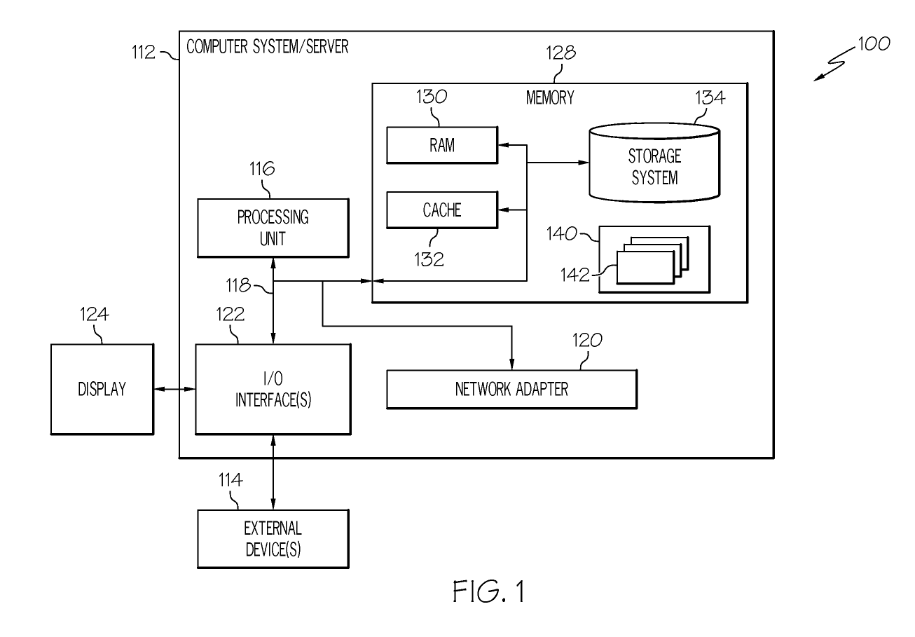

FIG. 1 is a block diagram of a data processing system in which various embodiments may be implemented. Data processing system 100 is one example of a suitable data processing system and is not intended to suggest any limitation as to the scope of use or functionality of embodiments of the invention described herein. Regardless, data processing system 100 is capable of being implemented and/or performing any of the functionality set forth herein.

In data processing system 100 there is a computer system/server 112, which is operational with numerous other general purpose or special purpose computing system environments, peripherals, or configurations. Examples of well-known computing systems, environments, and/or configurations that may be suitable for use with computer system/server 112 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 112 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 112 may be practiced in distributed computing environments where tasks are performed by remote processing devices that are linked through a communications network. In a distributed computing environment, program modules may be located in both local and remote computer system storage media including memory storage devices.

As shown in FIG. 1, computer system/server 112 in data processing system 100 is shown in the form of a general-purpose computing device. The components of computer system/server 112 may include, but are not limited to, one or more processors or processing units 116, a system memory 128, and a bus 118 that couples various system components including system memory 128 to processor 116.

Bus 118 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 112 typically includes a variety of computer system readable media. Such media may be any available media that is accessible by computer system/server 112, and it includes both volatile and non-volatile media, removable and non-removable media.

System memory 128 can include computer system readable media in the form of volatile memory, such as random access memory (RAM) 130 and/or cache memory 132. Computer system/server 112 may further include other removable/non-removable, volatile/non-volatile computer system storage media. By way of example, storage system 134 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 118 by one or more data media interfaces. Memory 128 may include at least one program product having a set (e.g., at least one) of program modules that are configured to carry out the functions of embodiments of the invention. Memory 128 may also include data that will be processed by a program product.

Program/utility 140, having a set (at least one) of program modules 142, may be stored in memory 128 by way of example, and not limitation, as well as an operating system, one or more application programs, other program modules, and program data. Each of the operating system, one or more application programs, other program modules, and program data or some combination thereof, may include an implementation of a networking environment. Program modules 142 generally carry out the functions and/or methodologies of embodiments of the invention. For example, a program module may be software for monitoring and tracking user interaction patterns to provide early warning of cognitive impairment.

Computer system/server 112 may also communicate with one or more external devices 114 such as a keyboard, a pointing device, a display 124, etc.; one or more devices that enable a user to interact with computer system/server 112; and/or any devices (e.g., network card, modem, etc.) that enable computer system/server 112 to communicate with one or more other computing devices. Such communication can occur via I/O interfaces 122 through wired connections or wireless connections. Still yet, computer system/server 112 can communicate with one or more networks such as a local area network (LAN), a general wide area network (WAN), and/or a public network (e.g., the Internet) via network adapter 120. As depicted, network adapter 120 communicates with the other components of computer system/server 112 via bus 118. It should be understood that although not shown, other hardware and/or software components could be used in conjunction with computer system/server 112. Examples, include, but are not limited to: microcode, device drivers, tape drives, RAID systems, redundant processing units, data archival storage systems, external disk drive arrays, etc.

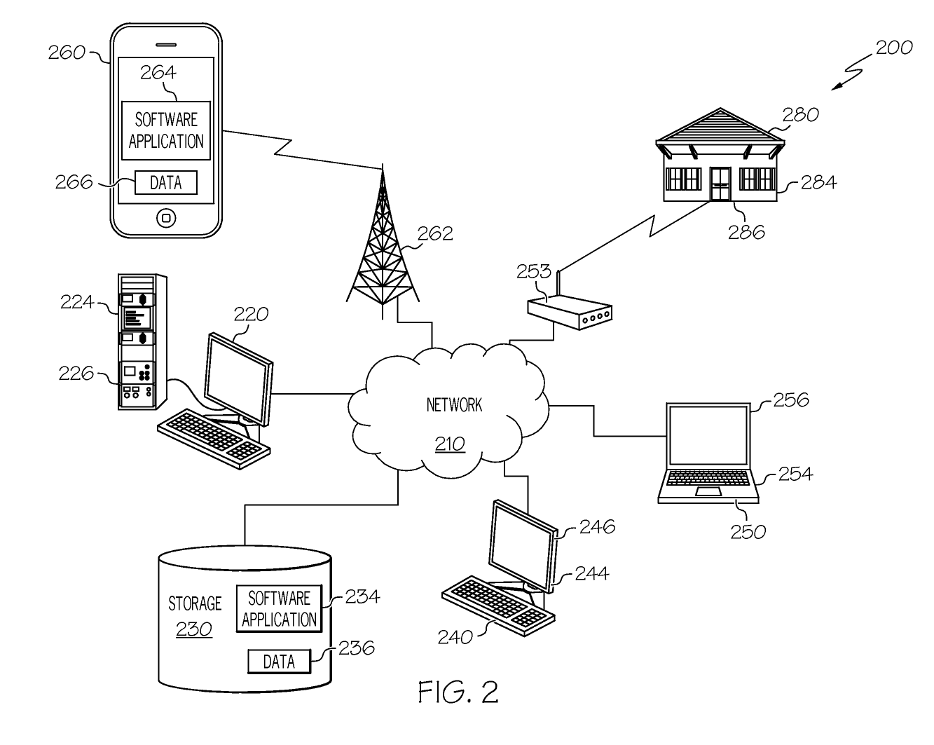

FIG. 2 is a block diagram of a network of data processing systems in which various embodiments may be implemented. Data processing environment 200 is a network of data processing systems such as described above with reference to FIG. 1. Software applications may execute on any computer or other type of data processing system in data processing environment 200. Data processing environment 200 includes network 210. Network 210 is the medium used to provide simplex, half duplex and/or full duplex communications links between various devices and computers connected together within data processing environment 200. Network 210 may include connections such as wire, wireless communication links, or fiber optic cables.

Server 220 and client 240 are coupled to network 210 along with storage unit 230. In addition, laptop 250 and facility 280 (such as a home or business) are coupled to network 210 including wirelessly such as through a network router 253. A mobile phone 260 may be coupled to network 210 through a mobile phone tower 262. Data processing systems, such as server 220, client 240, laptop 250, mobile phone 260 and facility 280 contain data and have software applications including software tools executing thereon. Other types of data processing systems such as personal digital assistants (PDAs), smartphones, tablets and netbooks may be coupled to network 210.

Server 220 may include software application 224 and data 226 for monitoring and tracking user interaction patterns to provide early warning of cognitive impairment or other software applications and data in accordance with embodiments described herein. Storage 230 may contain software application 234 and a content source such as data 236 for monitoring and tracking user interaction patterns to provide early warning of cognitive impairment. Other software and content may be stored on storage 230 for sharing among various computer or other data processing devices. Client 240 may include software application 244 and data 246. Laptop 250 and mobile phone 260 may also include software applications 254 and 264 and data 256 and 266. Facility 280 may include software applications 284 and data 286. Other types of data processing systems coupled to network 210 may also include software applications. Software applications could include a web browser, email, or other software application that can monitor and track user interaction patterns to provide early warning of cognitive impairment.

Server 220, storage unit 230, client 240, laptop 250, mobile phone 260, and facility 280 and other data processing devices may couple to network 210 using wired connections, wireless communication protocols, or other suitable data connectivity. Client 240 may be, for example, a personal computer or a network computer.

In the depicted example, server 220 may provide data, such as boot files, operating system images, and applications to client 240 and laptop 250. Server 220 may be a single computer system or a set of multiple computer systems working together to provide services in a client server environment. Client 240 and laptop 250 may be clients to server 220 in this example. Client 240, laptop 250, mobile phone 260 and facility 280 or some combination thereof, may include their own data, boot files, operating system images, and applications. Data processing environment 200 may include additional servers, clients, and other devices that are not shown.

In the depicted example, data processing environment 200 may be the Internet. Network 210 may represent a collection of networks and gateways that use the Transmission Control Protocol/Internet Protocol (TCP/IP) and other protocols to communicate with one another. At the heart of the Internet is a backbone of data communication links between major nodes or host computers, including thousands of commercial, governmental, educational, and other computer systems that route data and messages. Of course, data processing environment 200 also may be implemented as a number of different types of networks, such as for example, an intranet, a local area network (LAN), or a wide area network (WAN). FIG. 2 is intended as an example, and not as an architectural limitation for the different illustrative embodiments.

Among other uses, data processing environment 200 may be used for implementing a client server environment in which the embodiments may be implemented. A client server environment enables software applications and data to be distributed across a network such that an application functions by using the interactivity between a client data processing system and a server data processing system. Data processing environment 200 may also employ a service oriented architecture where interoperable software components distributed across a network may be packaged together as coherent business applications.

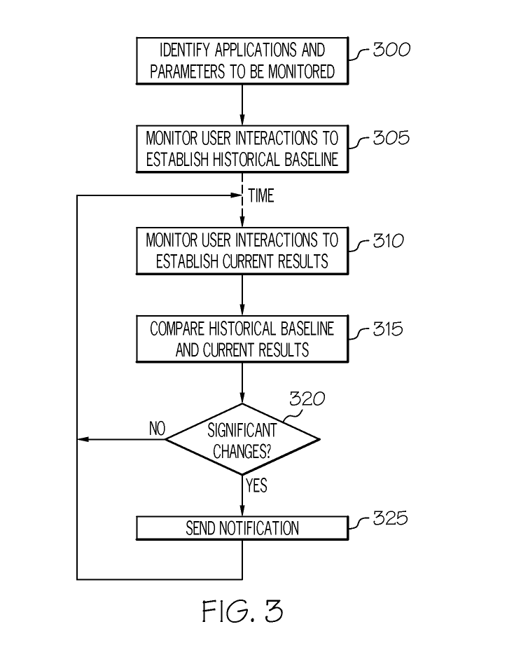

FIG. 3 is a high level flow diagram of user interaction monitoring in which various embodiments may be implemented. In a first step 300, applications and parameters to be monitored are identified. These may be identified by a health care professional or other responsible party and then downloaded to a local system(s) that the user interacts with. The applications on the local system(s) can then be monitored. In step 305, the results of that monitoring are used to establish an historical baseline for the user. After some period of time the applications on the local system(s) utilized by the user are again monitored in step 310. The results of that monitoring are then used to establish current results for the user in step 310. That can include observing trends in user interaction over time. Then in step 315, the historical baseline is compared with the current results. If significant changes are observed in step 320, then processing continues to step 325, otherwise processing returns to step 310. In step 325, a notification is sent to the user, a professional, a health care provider, a relative, or other designated recipient identified in a predetermined list, the notification including a description of the significant change and the supporting data. After the notification is sent, then processing returns to step 310.

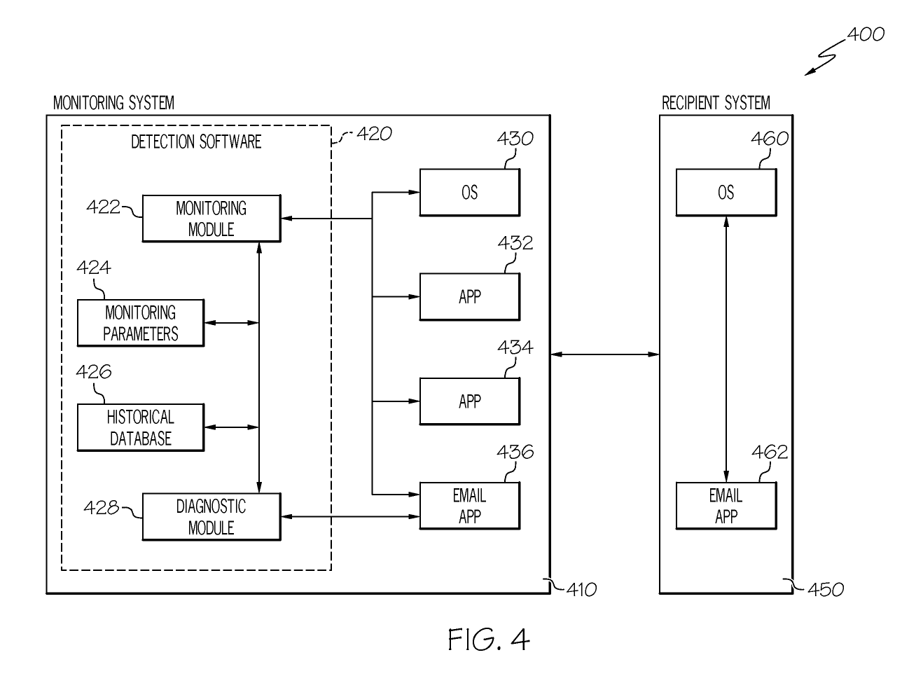

FIG. 4 is a block diagram of a single system monitoring implantation 400 in accordance with a first embodiment. A monitoring data processing system 410 includes a variety of software and data used for implementing the first embodiment such as a mobile device, a game device, a smart phone, a desktop computer, a laptop computer, and a server computer. Detection software 420 includes monitoring module 422, monitoring parameters 424, historical data 426, and diagnostic module 428. Detection software 420 is also in communication with various types of internal software such as operating system 430, a first application 432, a second application 434, and an email application 436. Monitoring system 410 is in communication with an external recipient data processing system 450. Recipient system 450 is used by a health care professional or other person designated as the receiver of any monitoring results. Recipient system 450 also includes an operating system 460 and an email application 462.

Monitoring module 422 has a low level connection with operating system 430. This allows the monitoring module to follow keyboard key strokes, mouse clicks, etc. of a user to determine whether there has been any degradation of user actions on those devices. This also allows the monitoring module to differentiate between different users of the data processing system assuming there is a log in process through the operating system.

Monitoring module 422 also connects with the application program interfaces (APIs) of various applications (apps) including application 432, application 434, and email application 436. This allows the monitoring module to monitor various actions of a user within those applications. For example, if application 432 is a document processing system or a texting application, the number of misspellings by a user detected by that application may be useful. For another example, if application 434 is a video game, the response time of a user may be useful. Many additional applications may be located on and monitored on data processing system 410.

Monitoring module 422 obtains data from the operating system and various applications in accordance with parameters set forth in monitoring parameters 424. These parameters may be a generic set of parameters or may be set by a physician or other health care provider based on any suspect cognition impairment. The results of this monitoring is then stored in historical database 426 for analysis by diagnostic module 428.

Diagnostic module 428 can review the historical database and determine whether there may be a change in user actions either indicating or contraindicating possible cognition impairment. Diagnostic module 428 can also summarize and/or erase certain historical database for managing the size of the historical database. The results of this diagnosis may be sent to the user, a professional, a health care provider, a relative or other designated recipient identified in a predetermined list through email application 436 to email application 462. Results could also be sent through a web browser application or other type of communication software. Results may also be provided directly to the user for self-reporting to a health care professional if needed.

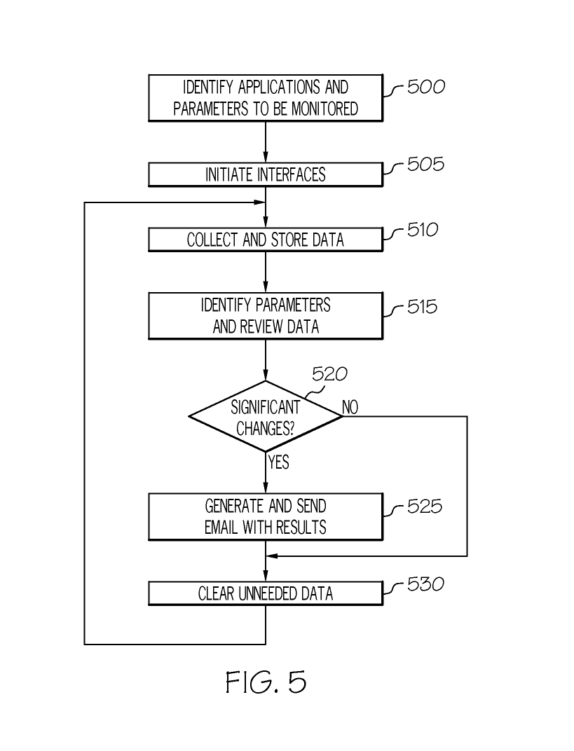

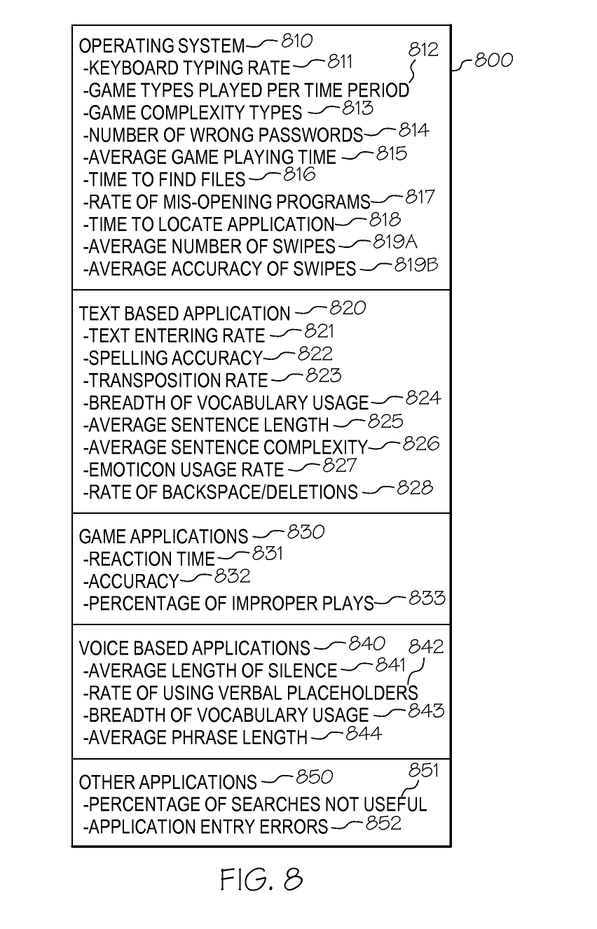

FIG. 5 is a flow diagram of the operation of the single system monitoring implementation in accordance with the first embodiment. This process can be initiated whenever the user's monitoring system is booted, when the user logs onto that system, or upon the occurrence of other events. In a first step 500, the monitoring module reads the monitoring parameters database to identify the applications and parameters to be monitored. An example of a monitoring parameters database is described below with reference to FIG. 8. Subsequently in step 505, the monitoring module initiates interfaces with the operating system and applications in accordance with the parameters to be monitored. Then in step 510, data is collected from monitored software and is stored in the historical database. This may be continuously updated, updated at certain intervals, or updated at certain events such as when the user logs off the data processing system.

Subsequently or concurrently with step 510, the diagnostic module reads the monitoring parameters database to identify the parameters being monitored and reviews the historical database in accordance with those monitoring parameters in step 515. This can occur at certain intervals or upon the occurrence of certain events such as when the user logs off the data processing system. Subsequently in step 520, the diagnostic module compares recent results against a baseline set previously (also stored in the historical database) and against more recent data to determine whether there have been any changes of significance. Significant changes could be a significant reduction in response time and spelling, or an ability to handle complex tasks such as in games or even in sentence structure. If any significant changes are identified in step 520, then processing continues to step 525, otherwise processing continues to step 530. In step 525, an email or other communication is generated and sent to the user, a professional, a health care provider, a relative, or other designated person identified in a predetermined list including a description of the significant changes and including the data supporting that determination. This will allow the recipient such as a health care professional to either make a diagnosis or request additional testing. In alternative embodiments, an email or other communication may be sent with supporting data even if no significant changes are diagnosed. In step 530, the diagnosis module can optionally clear any unneeded data from the historical database to reduce the amount of data stored in memory. Alternatively, all historical data may be retained for purposes of statistical analysis and/or comparison to future diagnostics. Processing can then return to step 510.

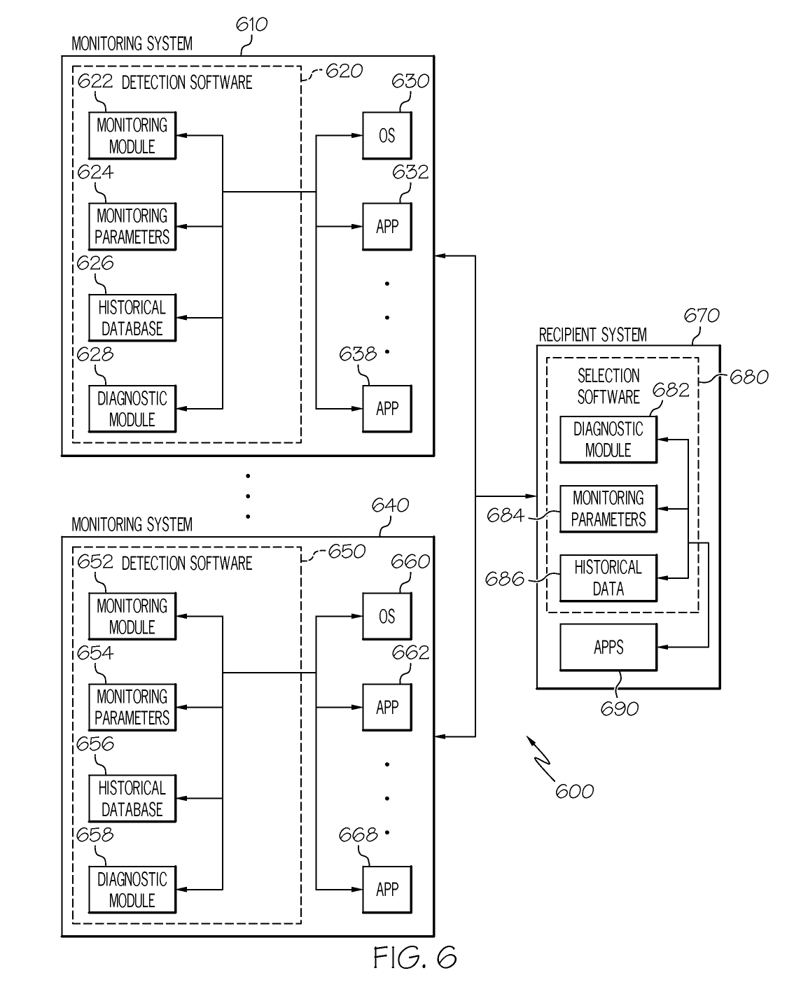

FIG. 6 is a block diagram of a multi-system implantation 600 in accordance with a second embodiment. This embodiment is aimed at monitoring the usage of multiple devices by a single user. However, multiple users may also be monitored using the same or similar type of system. Multiple monitoring data processing systems 610 and 640 includes a variety of software and data used for implementing the first embodiment. Monitoring system 610 may be a desktop computer utilized by the user, and monitoring system 640 may be a mobile phone or other device utilized by the user. Although two monitoring systems are shown, many other monitoring systems and types of monitoring systems may be included such as a mobile device, a game device, a smart phone, a desktop computer, a laptop computer, and a server computer.

Each monitoring system includes detection software 620 and 650 with a monitoring module 622 and 652, monitoring parameters 624 and 654, historical data 626 and 656, and a diagnostic module 628 and 658. Detection software 620 and 650 are also in communication with various types of internal software such as operating system 630 and 660, a first application 632 and 662 and a second application 638 and 668. Monitoring systems 610 and 640 are in communication with an external recipient data processing system 670. Recipient system 670 is used to collect and analyze data from multiple monitoring systems for the same user, and may be used by a health care professional or other person designated as the receiver of any monitoring results. Recipient system 670 includes diagnostic software 680 and multiple applications 690. Diagnostic software 680 includes a diagnostic module 682, monitoring parameters 684 and historical database 686. Recipient system 670 can be a remote system such as a server or it may be a virtual system located in a cloud environment.

Each monitoring module 622 and 652 has a low level connection with an operating system 630 or 660. This allows each monitoring module to follow keyboard key strokes, mouse clicks, etc. of a user on that system to determine whether there has been any degradation of user actions on those devices. This also allows each monitoring module to differentiate between different users of each monitoring system assuming there is a log in process through the operating system.

Each monitoring module 622 and 652 also connects with the application program interfaces (APIs) of various applications (apps) including application 632 or 662, or application 638 or 668. This allows the monitoring module to monitor various actions of a user within those applications. For example, if application 632 or 662 is a document processing system or a texting application, the number of misspellings by a user detected by that application may be useful. For another example, if application 638 or 668 is a video game, the response time of a user may be useful. Many additional applications may be located on and monitored on data processing systems 610 and 640.

Each monitoring module 622 and 652 obtains data from the corresponding operating system and various applications in accordance with parameters set forth in monitoring parameters 624 or 654. These parameters may be a generic set of parameters or may be set by a physician or other health care provider based on any suspect cognition impairment. The results of this monitoring is then stored in historical database 626 or 656 for analysis by diagnostic module 628 or 658.

Diagnostic module 628 and 658 can review the corresponding historical database and determine whether there may be a change in user actions either indicating or contraindicating possible cognition impairment. Diagnostic module 628 and 658 can also summarize and/or erase certain historical database for managing the size of the historical database. The results of this diagnosis may be sent to the user, a professional, a health care provider, a relative, or other designated recipient identified in a predetermined list. Results could also be sent through a web browser application or other type of communication software and should include identification information of the user being monitored. This identification information may be an identification number of the user that would preserve the confidentiality of that patient except to the approved caregiver. The results can include diagnostic results and supporting data. The results can also include raw data or cumulative data to allow a recipient system to review results from multiple monitoring systems.

Detection software 680 of recipient system 670 can review the diagnostic results of multiple monitoring systems for a single user or for multiple users. Monitoring parameters 684 is updated by a health care professional or other authorized person to indicate the applications to be monitored and data to be assessed and forwarded to historical database 686. If monitoring parameters database 684 is modified, then the changes may be downloaded to the monitoring systems for updating their respective monitoring parameters databases. Diagnostic module 682 can review the information collected in historical database 686 including any diagnosis, results, accumulations of data, and raw data. Diagnostic module can then make further assessments of this information to determine whether there are possible indicators of cognitive impairment based on the user's activities across multiple monitoring devices.

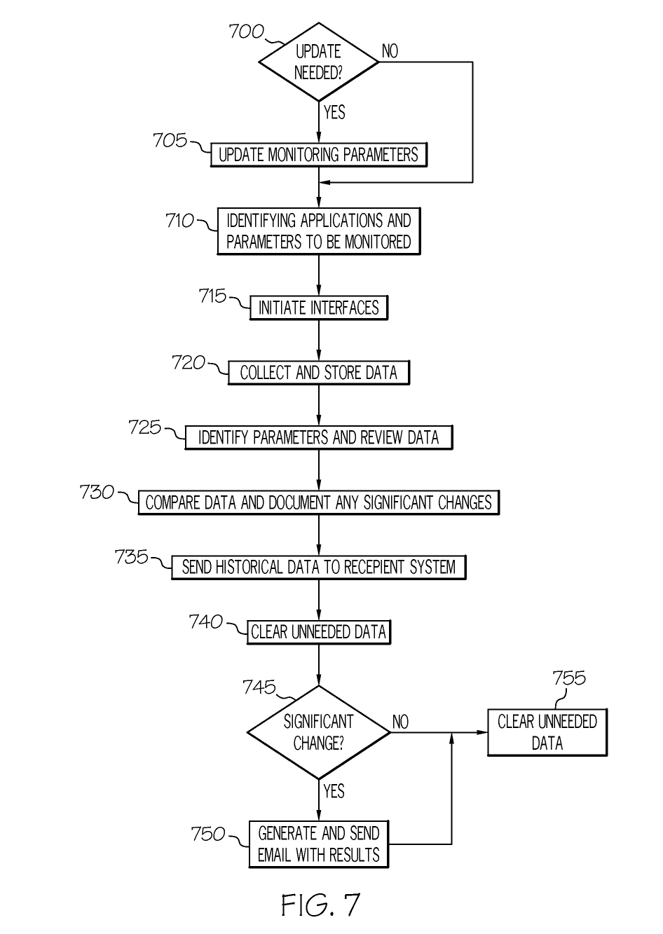

FIG. 7 is a flow diagram of the operation of the multi-system implementation in accordance with the second embodiment. This process can be initiated whenever any of the user's monitoring systems is booted, when the user logs onto a monitoring system, or upon the occurrence of other events. In a first step 700, the monitoring system detection software determines whether the local monitoring parameters database needs to be updated. The recipient system monitoring parameters database may have been updated by a health care professional or other authorized person, thereby changing which parameters should be monitored at the monitoring systems. This determination could be accomplished by inquiring the recipient system detection software or through other known techniques. This could be accomplished through an internet, Wi-Fi, cellular, or other wireless or wired connection between the monitoring system and the recipient system. If an update is needed, then processing continues to step 705, otherwise processing continues to step 710. In step 705, the monitoring parameters are updated from the monitoring parameters in the recipient system. Processing then continues to step 710.

In a step 710, the monitoring module reads the monitoring parameters database to identify the applications and parameters to be monitored. An example of a monitoring parameters database is described below with reference to FIG. 8. Subsequently in step 715, the monitoring module initiates interfaces with the operating system and applications in accordance with the parameters to be monitored. Then in step 720, data is collected from monitored software and is stored in the historical database. This may be continuously updated, updated at certain intervals, or updated at certain events such as when the user logs off the data processing system.

Subsequently or concurrently with step 720, the monitoring system diagnostic module reads the monitoring parameters database to identify the parameters being monitored and reviews the historical database in accordance with those monitoring parameters in step 725. This can occur at certain intervals or upon the occurrence of certain events such as when the user logs off the data processing system. Subsequently in step 730, the diagnostic module compares recent results against a baseline set previously (also stored in the historical database) and against more recent data to determine whether there have been any changes of significance. Significant changes could be a significant reduction in response time and spelling, or an ability to handle complex tasks such as in games or even in sentence structure. If a significant change is identified, then the monitoring system historical database is updated with a description of the significant change and including the data supporting that determination. Subsequently in step 735, information including collected data, accumulations, the description of any significant changes and related supporting data is then sent to the recipient system for storage in the recipient system historical database. This could be accomplished through an internet, Wi-Fi, cellular, or other wireless or wired connection between the monitoring system and the recipient system. Then in step 740, the diagnosis module then clears any unneeded data from the historical database to reduce the amount of data stored in memory.

Subsequently in step 745, the recipient system diagnostic module reads the monitoring parameters database to identify the parameters being monitored, reviews the historical database in accordance with those monitoring parameters, and compares recent results against a baseline set previously (also stored in the historical database) and against more recent data received from the monitoring systems to determine whether there have been any changes of significance. The diagnostic module can also utilize information obtained from multiple monitoring systems to confirm or not the significant changes identified by each individual monitoring system. Significant changes could be a significant reduction in response time and spelling, or an ability to handle complex tasks such as in games or even in sentence structure. If any significant changes are identified or confirmed in step 745, then processing continues to step 750, otherwise processing continues to step 755. In step 750, an email or other notification is generated and sent to the user, a professional, a health care provider, a relative, or other designated person identified in a predetermined list, the notification including a description of the significant changes and including the data supporting that determination. This will allow the health care professional to either make a diagnosis or request additional testing. In alternative embodiments, an email or other communication may be sent with supporting data even if no significant changes are diagnosed. Processing then continues to step 755. In step 755, the recipient system diagnosis module can optionally clear any unneeded data from the recipient system historical database to reduce the amount of data stored in memory. Alternatively, all historical data may be retained for purposes of statistical analysis and/or comparison to future diagnostics.

Although each monitoring system in this embodiment includes a diagnostic module, alternative embodiments may not include a diagnostic module in those monitoring systems. Instead, the monitoring modules may just forward monitored data to the recipient system where all diagnostics are performed. This may require the transmission or more data, but allows for simpler software at the monitoring system level.

FIG. 8 is a block diagram of a monitoring parameters database 800 in which various embodiments may be implemented. This is a list of possible candidates for monitoring user interactions in the areas of text, game, procedure, semantic, voice, and gestures, but other types of interactions may be monitored based on research or to do research. Some of these interactions are specific to smartphones while others may be specific to any type of data processing system. These types of interactions can be monitored and compared for threshold differences in vocabulary, sentence structure, playing time, game types, game complexity, errors, number of repeated searches, accuracy, quantity, duration, rate, contraction, emotion, navigation, voice place holders, selection errors, reaction time, and other changes.

A first set 810 of interactions to be monitored includes items that can be monitored with a low level connection with an operating system. This includes a keyboard typing rate 811, a number of game types played per time period 812 (e.g. moving from playing crosswords, jumble, sudoku, solitaire to just solitaire), a change in game complexity types 813 (from crosswords to word find), number of wrong passwords entered per tries 814, average amount of game playing time per time period 815, time taken to find files in a directory tree after parent node opened 816, rate of opening then immediately closing a program 817 (e.g. opening then closing a document program, then opening a spreadsheet program), time taken to locate an application on a smartphone 818 (e.g. flipping through phone screens to find an application), average number of smartphone swipes to execute an interaction 819A (e.g. taking multiple swipes to get a delete to show up), and average accuracy of swipes on a smartphone 819B (e.g. swiping wrong entry, the swiping correct entry to perform an action).

A second set 820 of interactions to be monitored includes items that can be monitored with an application program interface (API) connection to a text based application such as email, document processing, and text based applications. This includes text entering rate 821, spelling accuracy 822, letter transposition rate 823, breadth of vocabulary usage 824, average sentence length 825, average sentence complexity 826 (e.g. verb, noun, adjective count and conjunctions per sentence), emoticon usage rate 827 (e.g. usage rate of sad face emoticon or breadth of emoticon type usage), and rate of backspace and deletions of letters, words or sections 828.

A third set 830 of interactions to be monitored includes items that can be monitored with an API connection to various game applications. This includes reaction time 831 (e.g. time to fire at a target after the target appears), accuracy 832 (e.g. percentage of targets hit), and percentage of plays attempted that are improper 833 (e.g. attempting to put a red card on a red card in solitaire).

A fourth set 840 of interactions to be monitored includes items that can be monitored with an API connection to various voice based applications. This includes average length of periods of silence 841, rate of usage of verbal place holders 842 (e.g. umms, you knows, etc.), breadth of vocabulary usage 843, and average phrase length 844.

A fifth set 850 of interactions to be monitored includes items that can be monitored with an API connection to various other applications such as search engines, tax programs, etc. This includes a percentage of searches that were not useful 851 (e.g. enter search criteria but does not check any of the results) and application entry errors 852 (e.g. errors returned by checking routines such as in a tax calculation program).

The invention can take the form of an entirely software embodiment, or an embodiment containing both hardware and software elements. In a preferred embodiment, the invention is implemented in software or program code, which includes but is not limited to firmware, resident software, and microcode.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, microcode, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM), or Flash memory, an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Further, a computer storage medium may contain or store a computer-readable program code such that when the computer-readable program code is executed on a computer, the execution of this computer-readable program code causes the computer to transmit another computer-readable program code over a communications link. This communications link may use a medium that is, for example without limitation, physical or wireless.

A data processing system suitable for storing and/or executing program code will include at least one processor coupled directly or indirectly to memory elements through a system bus. The memory elements can include local memory employed during actual execution of the program code, bulk storage media, and cache memories, which provide temporary storage of at least some program code in order to reduce the number of times code must be retrieved from bulk storage media during execution.

A data processing system may act as a server data processing system or a client data processing system. Server and client data processing systems may include data storage media that are computer usable, such as being computer readable. A data storage medium associated with a server data processing system may contain computer usable code such as for monitoring and tracking user interaction patterns to provide early warning of cognitive impairment. A client data processing system may download that computer usable code, such as for storing on a data storage medium associated with the client data processing system, or for using in the client data processing system. The server data processing system may similarly upload computer usable code from the client data processing system such as a content source. The computer usable code resulting from a computer usable program product embodiment of the illustrative embodiments may be uploaded or downloaded using server and client data processing systems in this manner.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to explain the principles of the invention, the practical application, and to enable others of ordinary skill in the art to understand the invention for various embodiments with various modifications as are suited to the particular use contemplated.

The terminology used herein is for the purpose of describing particular embodiments and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method of monitoring user interaction patterns to provide early warning of cognitive impairment comprising:
monitoring with an application running on a processor a first set of user interactions of a user with a data processing system to form a historical baseline for the user that is stored in memory;
monitoring with the application running on the processor a second set of user interactions of the user with the data processing system to form a set of current results for the user;
comparing the set of current results with the historical baseline; and
responsive to identifying a change indicative of a cognitive degradation for the user, sending a notification indicating the change.

2. The method of claim 1 wherein the historical baseline and the current set of results are based on a set of parameters stored in memory.

3. The method of claim 2 wherein the set of parameters may be updated remotely.

4. The method of claim 1 wherein the monitoring may be performed across multiple data processing systems, each system sending any results of monitoring to a central data processing system, the central system performing the comparing.

5. The method of claim 1 wherein the historical baseline and the current set of results are based on a set of parameters stored in memory; wherein the set of parameters may be updated remotely; wherein the monitoring may be performed across multiple data processing systems, each system sending any results of monitoring to a central data processing system, the central system performing the comparing; wherein the notification is sent by email; and wherein an unneeded data is cleared from memory, and wherein the multiple data processing systems are selected from a group consisting of a mobile device, a game device, a smartphone, a desktop computer, a laptop computer, and a server computer.

6. The method of claim 1 wherein the data processing system is selected from a group consisting of a mobile device, a game device, a smartphone, a desktop computer, a laptop computer, and a server computer; and wherein the first set of user interactions is selected from a group consisting of text, game, procedure, semantic, voice, and gestures.

7. The method of claim 6 wherein the change is a threshold difference selected from a group consisting of vocabulary, sentence structure, playing time, game types, game complexity, errors, number of repeated searches, accuracy, quantity, duration, rate, contraction, emotion, navigation, voice place holders, selection errors, and a reaction time.

8. The method of claim 7 wherein the notification is selected from a group consisting of the user, a professional, a health care provider, a relative, and other designated person identified in a predetermined list.

9. A computer usable program product comprising a computer usable storage medium including computer usable code for use in monitoring user interaction patterns to provide early warning of cognitive impairment, the computer usable program product comprising code for performing the steps of:
monitoring with an application running on a processor, a first set of user interactions of a user with a data processing system to form a historical baseline for the user that is stored in memory;
monitoring with the application running on the processor a second set of user interactions of the user with the data processing system to form a set of current results for the user;
comparing with the processor the set of current results with the historical baseline; and
responsive to identifying a change indicative of a cognitive degradation for the user, sending a notification indicating the change.

10. The computer usable program product of claim 9 wherein the historical baseline and the current set of results are based on a set of parameters stored in memory wherein the set of parameters may be updated remotely.

11. The computer usable program product of claim 9 wherein the monitoring may be performed across multiple data processing systems, each system sending any results of monitoring to a central data processing system, the central system performing the comparing.

12. The computer usable program product of claim 9 wherein the data processing system is selected from a group consisting of a mobile device, a game device, a smartphone, a desktop computer, a laptop computer, and a server computer.

13. The computer usable program product of claim 12 wherein the first set of user interactions is selected from a group consisting of text, game, procedure, semantic, voice, and gestures, and wherein the change is a threshold difference selected from a group consisting of vocabulary, sentence structure, playing time, game types, game complexity, errors, number of repeated searches, accuracy, quantity, duration, rate, contraction, emotion, navigation, voice place holders, selection errors, and reaction time.

14. The computer usable program product of claim 9 wherein the notification is selected from a group consisting of the user, a professional, a health care provider, a relative, and other designated person identified in a predetermined list.

15. A data processing system for monitoring user interaction patterns to provide early warning of cognitive impairment, the data processing system comprising:
a processor; and
a memory storing program instructions which when executed by the processor execute the steps of:
monitoring with an application running on the processor, a first set of user interactions of a user with a data processing system to form a historical baseline for the user that is stored in the memory;
monitoring with the application running on the processor a second set of user interactions of the user with the data processing system to form a set of current results for the user;
comparing with the processor the set of current results with the historical baseline; and
responsive to identifying a change indicative of a cognitive degradation for the user, sending a notification indicating the change.

16. The computer usable program product of claim 15 wherein the historical baseline and the current set of results are based on a set of parameters stored in memory wherein the set of parameters may be updated remotely.

17. The data processing system of claim 15 wherein the monitoring may be performed across multiple data processing systems, each system sending any results of monitoring to a central data processing system, the central system performing the comparing.

18. The data processing system of claim 15 wherein the data processing system is selected from a group consisting of a mobile device, a game device, a smartphone, a desktop computer, a laptop computer, and a server computer.

19. The data processing system of claim 18 wherein the first set of user interactions is selected from a group consisting of text, game, procedure, semantic, voice, and gestures, and wherein the change is a threshold difference selected from a group consisting of vocabulary, sentence structure, playing time, game types, game complexity, errors, number of repeated searches, accuracy, quantity, duration, rate, contraction, emotion, navigation, voice place holders, selection errors, and reaction time.

20. The data processing system of claim 15 wherein the notification is selected from a group consisting of the user, a professional, a health care provider, a relative, and other designated person identified in a predetermined list.
